(19) United States
(12) Patent Application Publication
(10) Pub. No.: US 2015/0250383 A1
(43) Pub. Date: Sep. 10, 2015

(54) REAL TIME VISUALIZATION OF SURGICAL OPERATIONS ON THE EYE

(71) Applicant: Annidis Corporation, Kanata (CA)

(72) Inventors: Zeljko RIBARIC, Kanata (CA); David A. KAHN, Kanata (CA)

(73) Assignee: Annidis Corporation, Kanata (CA)

(21) Appl. No.: 14/639,202

(22) Filed: Mar. 5, 2015

Related U.S. Application Data
(60) Provisional application No. 61/948,353, filed on Mar. 5, 2014.

(51) Int. Cl. A61B 3/10; A61B 3/12

(52) U.S. Cl. CPC A61B 3/102; A61B 3/12

(57) ABSTRACT

A real time stereo fundus viewer is disclosed which uses IR illumination introduced either corneally or through the sclera, arranged for frontal or rear illumination of the posterior regions of the eye and displayed using two miniature false colour displays; with the option of OCT.

This application claims priority based on U.S. Patent Application No. 61/948,353 entitled “REAL TIME VISUALIZATION OF SURGICAL OPERATIONS ON THE EYE” filed Mar. 5, 2014, which is herein incorporated by reference.

### BACKGROUND

Surgeons, in the course of their surgical operations, require high-quality real-time visualization to enable them to apply their tools at a suitable carnal location and in a suitable manner. Such visualization is preferably stereoscopic in order to present an appreciation of depth at the surface under treatment.

Surgical operations within the eye (ophthalmic operations), particularly in the posterior region that includes the retina and the choroid, present visualization challenges as the posterior tissues heavily absorb visible light. Moreover, the amount of light that can be applied to the posterior region is limited by patient safety and comfort issues.

The invention to be described addresses these challenges.

### THE INVENTION

It is well known that the optical absorption in the retinal and choroidal regions is far less in the infrared region of the spectrum than it is in the visible region. Advanced fundus cameras such as the Annidis RHA (U.S. Pat. No. 8,491,120 Retinal Fundus Surveillance Method and Apparatus, US 2013/030 1004 A1 pending—CIP, the disclosures of which are hereby incorporated by reference) illuminate using a range of spectra including those in the spectral region of 780 nm to 950 nm, usually but not necessarily generated by LEDs. Such illumination enables high quality imagery of the posterior regions when the image is collected by an image sensor that is sensitive to optical power at such wavelengths. Suitable image sensors include CCD and CMOS arrays that can capture detailed images that are made up of many megapixels, typically in the range of 1 to 10. These images can be displayed using conventional digital displays where the intensity of the infrared light captured at each pixel is represented by a visible colour that may be white or any part of the colour gamut visible to the human eye.

The Annidis RHA captures images using several bands of infrared illumination, as each different band is absorbed differently and the resulting images are able to be used to better identify different ocular pathologies that are not evident under visible illumination.

A good example is the visualization of melanin in the retinal pigment epithelium. Another example is the observation of surface adhesions.

Another feature that is enabled by the use of infrared light is the illumination of the posterior region through optical pathways other than the conventional pathway through the cornea and pupil of the eye. These other pathways are substantially opaque to visible light. As also featured in the Annidis RHA (US 2013/010 7211 A1 Pending—Method and Apparatus for Imaging the Choroid, the disclosure of which is hereby incorporated by reference), the illumination can be applied through the sclera, the white tissue surrounding the eyeball. There are two trans-scleral paths that can be used, each having useful features. The light may pass through the sclera near the front of the eye and then proceed to illuminate the front of the retina and choroid. Alternatively, the light may travel at length through the sclera, it acting as a waveguide, and finally being scattered from the sclera at the rear of the choroid, providing a rear illumination view of the posterior regions. Which of these two alternatives applies depends on the location and angular properties of the illumination reaching the sclera, as described in our application US 2013/010 7211 A1.

The Annidis RHA also features a stereoscopic capability. This is achieved by the use of a movable mask that blocks either one side or the other side of the light coming from the eye corresponding to two angles of view. This can be adapted such that each perspective is applied to a different image sensor, one associated with the right hand perspective and the other with a left-hand perspective.

The final step required to convert the images and present them in a form suitable for use by the surgeon, is to create the two stereo images in a miniature viewer that appears identical to the normal stereo viewer used by the surgeon. The difference is that the surgeon no longer sees a real image of the flesh under treatment but a reproduced false colour image presented on a miniature display in association with a suitable viewing lens. Such a miniature display can use an Organ Light-Emitting Diode (OLED) technology such as is used in products of eMagin Corp.

The overall result is that the surgeon is able to view in full false-colour stereo, images created using invisible infrared illumination that may or may not be introduced through the pupil, the latter being convenient in that there is no impediment to viewing directly through the pupil.

The implementation may also be augmented to include an Optical Coherence Tomography (OCT) function to give further precision to the operation. The OCT operates in the infrared and will not cause glare or damage to the patient. Its results may also be displayed on the OLED based viewer using a false colour.

The embodiments of the present disclosure are intended to be examples only. Those of skill in the art may affect alterations, modifications and variations to the particular embodiments without departing from the intended scope of the invention. The subject matter described herein and in the recited claims intends to cover and embrace all suitable changes in technology.

1. A real time stereo fundus viewer using IR illumination introduced either corneally or through the sclera, arranged for frontal or rear illumination of the posterior regions of the eye and displayed using two miniature false colour displays

2. The viewer of claim 1, further comprising an Optical Coherence Tomography (OCT) function, the results of which are displayed on at least one of the false colour displays.
